(19) United States
(12) Patent Application Publication
(10) Pub. No.: US 2005/0245788 A1
(43) Pub. Date: Nov. 3, 2005

(54) Esophageal delivery system and method with position indexing

(75) Inventor: Martin T. Gerber, Maple Grove, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/833,775

(22) Filed: Apr. 28, 2004

(51) Int. Cl. A61B 18/18; A61B 1/00; A61B 1/04; A61B 18/04

(52) U.S. Cl. 600/115; 606/27

(57) ABSTRACT

An esophageal delivery system includes features that facilitate the precise positioning of a medical device within the gastrointestinal tract. The system supports indexed positioning without relying solely on endoscopic viewing or external imaging equipment to identify the location of the medical device. A fixation element holds an elongated delivery device at a selected position within the gastrointestinal tract. The medical device is carried at a position that is a fixed and known distance away from the fixation element. Once the fixation element is positioned at a known location, precise positioning of the medical device within the esophagus can be achieved with greater certainty. As an illustration, the fixation element may take the form of a balloon an expandable frame or other device capable of engaging the lower esophageal sphincter (LES) to provide a reference position for a medical device to be placed at a precise distance above the LES.

### FIELD OF THE INVENTION

The invention relates to techniques for temporary deployment of medical devices in the esophagus and, more particularly, techniques for delivery of sensing or therapeutic devices to particular locations within the esophagus.

### BACKGROUND

Gastroesophageal reflux occurs when stomach fluid, which typically includes stomach acids, intermittently flows from the stomach into the esophagus. It is common for most people to experience this fluid reflux occasionally as heartburn. Gastroesophageal reflux disease (GERD) is a clinical condition in which the reflux of stomach fluid into the esophagus is frequent enough and severe enough to impact a patient's normal functioning or cause damage to the esophagus.

In the lower part of the esophagus, where the esophagus meets the stomach, there is a muscular valve called the lower esophageal sphincter (LES). Normally, the LES relaxes to allow food to enter into the stomach from the esophagus. The LES then contracts to prevent stomach fluids from entering the esophagus. In GERD, the LES relaxes too frequently or at inappropriate times, allowing stomach fluids to reflux into the esophagus.

The most common symptom of GERD is heartburn. Acid reflux may also lead to esophageal inflammation, which causes symptoms such as painful swallowing and difficulty swallowing. Pulmonary symptoms such as coughing, wheezing, asthma, or inflammation of the vocal cords or throat may occur in some patients. More serious complications from GERD include esophageal ulcers and narrowing of the esophagus. The most serious complication from chronic GERD is a condition called Barrett's esophagus in which the epithelium of the esophagus is replaced with abnormal tissue. Barrett's esophagus is a risk factor for the development of cancer of the esophagus.

Accurate diagnosis of GERD is difficult but important. Accurate diagnosis allows identification of individuals at high risk for developing the complications associated with GERD. It is also important to be able to differentiate between gastroesophageal reflux, other gastrointestinal conditions, and various cardiac conditions. For example, the similarity between the symptoms of a heart attack and heartburn often lead to confusion about the cause of the symptoms.

Esophageal manometry, esophageal endoscopy, and esophageal pH monitoring are standard methods of measuring esophageal exposure to stomach acids and are currently used to diagnose GERD. A variety of endoscopic devices have been designed to monitor various parameters within the esophagus. Many devices require an indwelling catheter to maintain a monitor in place within the esophagus.

The Bravo™ pH monitoring system, commercially available from Medtronic, Inc., of Minneapolis, Minn., is an example of a system useful in diagnosing GERD without the need for an indwelling catheter. The Bravo system includes an intra-luminal capsule that is temporarily placed within the esophagus via an endoscopic delivery device. The capsule has a vacuum cavity that captures a portion of the esophageal mucosal tissue. A physician then advances a pin through the captured tissue to secure the capsule relative to the esophageal wall. Eventually, the captured tissue sloughs away and releases the capsule, which then passes through the patient's gastrointestinal tract for eventual discharge. An example of a pH monitoring system is described in U.S. Pat. No. 6,689,056 to Kilcoyne et al., entitled “Implantable Monitoring Probe.”

When GERD is diagnosed, different therapy options are available to treat the condition. One therapy option for GERD is the administration of pharmaceutical agents to alter the pH of the stomach contents. Other GERD therapy options involve surgical or endoscopic repair of tissue in the region of the LES. For example, some techniques involve the use of an endoscopically delivered heating element to shrink and tighten tissue in the vicinity of the LES to enhance the structural integrity of the LES and thereby promote sustained closure.

Other GERD therapy options involve the implantation of bulking devices within the esophageal wall. The bulking device is implanted below the mucosal lining of the esophagus, and serves to enhance the residual closing pressure function of the sphincter so as to effectively reduce or prevent the reflux of stomach contents into the esophagus. An example of a bulking device is described in U.S. Pat. No. 6,401,718 to Johnson et al., entitled “Submucosal esophageal bulking device.”

Table 1 below lists documents that disclose various techniques for diagnosing or treating GERD.

TABLE 1

| Patent Number | Inventors | Title |
|---|---|---|
| 6,604,004 | Zelickson et al. | Device and method for treatment of |
| | | gastroesophageal reflux disease |
| 6,673,070 | Edwards et al. | Sphincter Treatment Apparatus |
| 6,285,897 | Kilcoyne et al. | Remote Physiological |
| | | Monitoring System |
| 6,689,056 | Kilcoyne et al. | Implantable Monitoring Probe |
| 6,251,063 | Silverman et al. | Method for treating wall forming |
| | | gastrointestinal tract |
| 6,401,718 | Johnson et al. | Submucosal esophageal bulking device |

All documents listed in Table 1 above are hereby incorporated by reference herein in their respective entireties. As those of ordinary skill in the art will appreciate readily upon reading the Summary of the Invention, Detailed Description of the Preferred Embodiments and claims set forth below, many of the devices and methods disclosed in the patents of Table 1 may be modified advantageously by using the techniques of the present invention.

### SUMMARY OF THE INVENTION

In general, the invention is directed to techniques for esophageal delivery of medical devices to precise locations within the esophagus. In some embodiments, the medical devices may be configured for diagnosis or treatment of GERD or other disorders, and may comprise monitors, stimulators, bulking devices, surgical devices, or other devices. An esophageal delivery system in accordance with the invention incorporates an expandable fixation element that permits precise positioning of the medical device relative to an esophageal feature such as the LES. In other embodiments, the invention may be configured to achieve precise positioning in the vicinity of the pyloric sphincter. In either case, the fixation element supports “indexed” positioning of the medical device. In particular, positioning is “indexed” in the sense that the medical device is carried at a position that is a fixed and known distance from the fixation element. In turn, the fixation element is known to be positioned proximate a structural feature such as the LES or pyloric sphincter.

Various embodiments of the present invention provide solutions to one or more problems existing in the prior art with respect to prior systems for esophageal delivery of medical devices. These problems include the inability of existing esophageal delivery systems to achieve precise positioning of the medical device relative to desired esophageal features such as the LES. Existing esophageal delivery systems typically require endoscopic visualization or external imaging to position the medical device within the esophagus. Unfortunately, endoscopic visualization can suffer from obscured viewing and limited viewing angles. External viewing creates undesirable complications due to the indirect nature of the view, and the need for access to fluoroscopic, ultrasound, or other imaging equipment. Even with these viewing techniques, precise positioning can be uncertain and requires signficant skill. If a medical device is not precisely positioned, the results of diagnosis or therapy may be inaccurate or ineffective, compromising the efficacy of the procedure for the patient. Consequently, existing techniques for esophageal delivery of medical devices suffer from inaccurate positioning, added time, and inconvenience, and and require extensive training.

Various embodiments of the present invention are capable of solving at least one of the foregoing problems. When embodied in a device for esophageal delivery of a medical device, for example, the invention includes a variety of features that facilitate the precise positioning of a medical device. In this manner, a physician need not rely solely on endoscopic viewing or external imaging equipment. The invention provides features that permit indexed positioning of a medical device relative to a fixation element carried by an esophageal delivery system. Once the fixation element is positioned at a known location within the esophagus, precise positioning of the medical device within the esophagus can be achieved with greater certainty. Accordingly, an esophageal delivery system in accordance with the invention may eliminate one or more of the problems that can result from uncertain and complicated positioning techniques that rely on endoscopic viewing or external imaging equipment.

Various embodiments of the invention may possess one or more features to solve the aforementioned problems in the existing art. In some embodiments, an esophageal delivery system may include an elongated delivery device sized for introduction into an esophagus of a patient, and a fixation element disposed adjacent a distal end of the elongated delivery device. The fixation element engages a first selected portion of the esophagus and thereby fixes the elongated delivery device against substantial upward movement within the esophagus. A deployment point is formed in the elongated delivery device at a fixed position relative to the fixation element such that the medical device is positioned adjacent a second selected portion of the esophagus. A medical device is deployed at the deployment point.

As an illustration, the fixation element may be expandable to a size that is larger than a passage defined by the LES. In this manner, according to an embodiment of an esophageal delivery method, the fixation element may be placed below the LES and then pulled upward to engage the LES, so that the medical device can be indexed to the LES and thereby placed at a precision position within the esophagus.

The fixation element may be an expandable balloon, an expandable frame, one or more expandable tines, or any of a variety of other expandable structures. In each case, the fixation element prevents substantial upward movement of the elongated delivery device so that the medical device can be placed precisely relative to the LES. The medical device may be, for example, a gastro-esophageal reflux monitor, a manometry sensor, a device for introducing a bulking agent into a wall of the esophagus, a heating element to heat tissue adjacent a lower esophageal sphincter of the patient, an electrical stimulator or a drug delivery device.

In other embodiments, the delivery device may be configured to permit placement of a medical device relative to the pyloric sphincter. In this case, the fixation element may be placed above the pyloric sphincter within the stomach, and the medical device can be placed below the pyloric sphincter within the small intestine. Accordingly, the medical device can be placed at a precise position relative to the pyloric sphincter, which serves as a reference position to index the position of the fixation element and the medical device.

In comparison to known techniques for placement of medical devices within the esophagus, or elsewhere in the gastrointestinal tract, various embodiments of the invention may provide one or more advantages. For example, the invention facilitates quick, convenient and accurate placement of a medical device within the esophagus. Positioning that is indexed to a known position within the esophagus, such as the LES, permits placement of the medical device at a precise location within the esophagus with a greater degree of certainty. In some cases, the invention may eliminate the need for endoscopic viewing or external imaging, or at least provide a more accurate placement technique that can be confirmed by viewing or imaging. In addition to greater precision, the time required for placement of the medical device may be reduced, resulting in a shorter procedure and possibly less patient discomfort. With more precise placement, the medical device is more likely to yield efficacious results. In addition, the invention may reduce the level of skill necessary to place a medical device, possibly permitting the placement procedure to be performed by physician's assistants, nurses, or other medical personnel other than a physician.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an esophageal delivery system for deployment of a medical device in the esophagus, shown in conjunction with a patient.

FIG. 2 is a schematic diagram illustrating placement of a distal fixation balloon of an endoscopic delivery device.

FIG. 3 is a schematic diagram illustrating placement of a distal fixation frame of an endoscopic delivery device.

FIG. 4 is a schematic diagram illustrating placement of distal fixation tines of an endoscopic delivery device.

FIG. 5 is a schematic diagram illustrating placement of a distal fixation balloon of an endoscopic delivery device carrying a monitor.

FIG. 6 is a schematic diagram illustrating placement of a distal fixation balloon of an endoscopic delivery device carrying a detachable monitor.

FIG. 7 is a schematic diagram illustrating the monitor of FIG. 6 upon detachment from the endoscopic delivery device.

FIG. 8 is a schematic diagram illustrating placement of a distal fixation balloon of an endoscopic delivery device carrying components for implantation of bulking agents in the esophageal wall.

FIG. 9 is a schematic diagram of the device of FIG. 8 following implantation of bulking agents in the esophageal wall.

FIG. 10 is a schematic diagram illustrating placement of a distal fixation balloon of an endoscopic delivery device carrying components for heat treatment of esophageal tissue.

FIG. 11 is a schematic diagram illustrating an esophageal delivery system for deployment of a medical device in the small intestine proximate the pyloric sphincter, shown in conjunction with a patient.

FIG. 12 is a schematic diagram illustrating placement of a distal fixation balloon of an endoscopic delivery device proximate the pyloric sphincter.

FIG. 13 is a flow diagram illustrating a method for placement of a medical device within the esophagus.

FIG. 14 is a flow diagram illustrating a method for placement of a medical device within the small intestine adjacent the pyloric sphincter.

### DESCRIPTION OF THE PREFERRED EMBODIMENTS

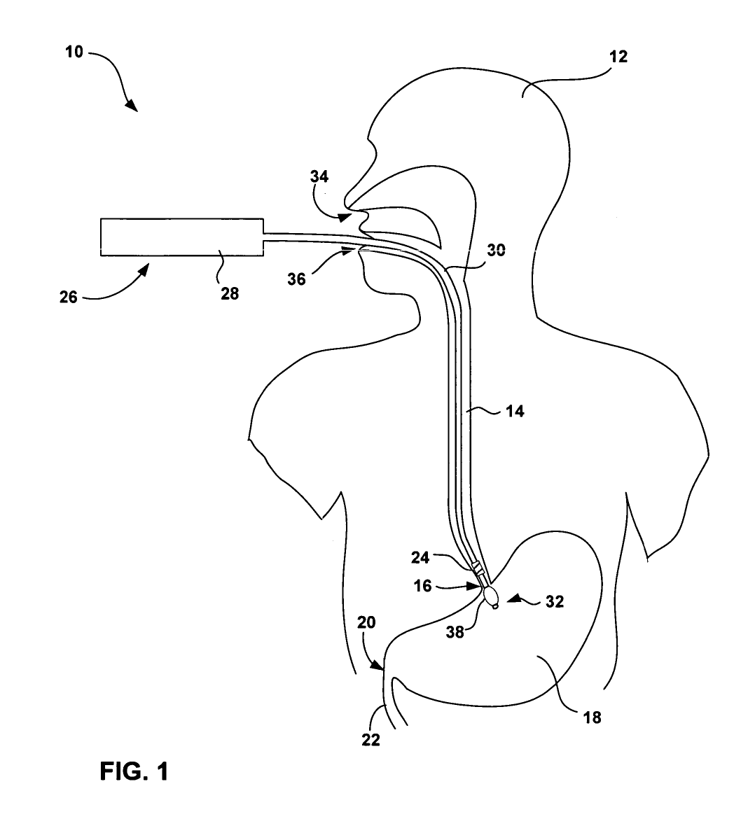

FIG. 1 is a schematic diagram illustrating an esophageal delivery system 10 shown in conjunction with a patient 12. As will be described, esophageal delivery system 10 is configured for esophageal delivery of any of a variety of medical devices to precise locations within an esophagus 14 of patient 12. The medical devices delivered by esophageal delivery system 10 may be configured for diagnosis or treatment of GERD, and may comprise monitors, stimulators, drug delivery devices, bulking devices, thermal delivery elements, surgical devices, or other devices.

In accordance with the invention, esophageal delivery system 10 incorporates an expandable fixation element that permits precise positioning of the medical device relative to an esophageal feature such as the lower esophageal sphincter (LES) 16 at the entrance to stomach 18. In other embodiments, esophageal delivery system 10 may be configured to achieve precise positioning in the vicinity of pyloric sphincter 20 at the entrance to small intestine 22.

As shown in FIG. 1, esophageal delivery system 10 serves to position and place a medical device 24 within the gastrointestinal tract of patient 12. Esophageal delivery system 10 includes an endoscopic delivery device 26 having a proximal portion, referred to herein as a handle 28, and a flexible probe 30 that extends from handle 28 into the gastrointestinal tract of patient 12. Medical device 24 is coupled adjacent a distal end 32 of delivery device 26 for delivery to a target location within the esophagus 14. Distal end 32 of delivery device 26 enters esophagus 14, via either nasal cavity 34 or oral cavity 36, and extends into esophagus 14 to a desired placement location.

An expandable fixation element 38 is disposed adjacent distal end 32 of endoscopic delivery device 26. In the example of FIG. 1, expandable fixation element 38 is an expandable balloon that extends radially outward from flexible probe 30 upon inflation with liquid or gas. Upon deployment of distal end 32 of endoscopic delivery device 26 into stomach 18, fixation element 38 is expanded to thereby anchor flexible probe 30 against substantial upward movement.

Fixation element 38, upon expansion, is sized larger than a passage defined by LES 16. Medical device 24 is positioned at a depolyment point at a known, fixed distance from fixation element 38, on a side of the fixation element opposite the distal tip of flexible probe 30. In this manner, when fixation element 38 engages LES 16 to oppose upward movement of probe 30, the LES provides an indexing or “reference” point for precise positioning of medical device 24. Accordingly, a physician may pull upward on probe 30 to engage fixation element 38 against LES 16 and anchor probe 30, and then deploy or activate medical device 24 for use within esophagus 14.

Medical device 24 may be designed for use as part of flexible probe 30, i.e., on an in-dwelling basis. In this case, probe 30 carries medical device 24 and holds the medical device in place at a precise position. In other cases, medical device 24 may be designed for detachment from flexible probe 30 and attachment within or to the mucosal lining of esophagus 14 or LES 16. Medical device 24 is represented generally in FIG. 1, and may take a variety of different forms.

Precise positioning of medical device 24 is important for different diagnostic and therapeutic applications. Examples include placement of a pH monitor adjacent LES 16 for GERD diagnosis, placement of a manometry sensor for pressure readings adjacent LES 16, placement of a flow meter for flow readings adjacent LES 16, placement of a bulking device within or proximate LES 16 to treat GERD, placement of a surgical or thermal heating device at a precise position relative to LES 16 to treat tissue in the vicinity of the LES, and placement of an electrical stimulator or drug delivery device adjacent LES 16 to modify physiological activity of the gastrointestinal tract within esophagus 14 or LES 16. In each case, expandable fixation element 38 and the fixed distance between medical device 24 and the fixation element permit ready positioning of the medical device at a precise location.

Precise positioning of medical device 24 may be aided by endoscopic viewing provided by an imaging endoscope integrated within or delivered simultaneously with flexible probe 30. In addition, external imaging techniques such as fluoroscopy or ultrasonic imaging may be used to aid precise positioning of medical device 24. However, indexed positioning relative to expandable fixation element 38 provides a very quick, convenient, and accurate mechanism for placement of medical device 24 at an initial position. In this manner, the procedure for placement of medical device 24 may be simplified, reducing the time necessary for placement and possibly reducing patient discomfort.

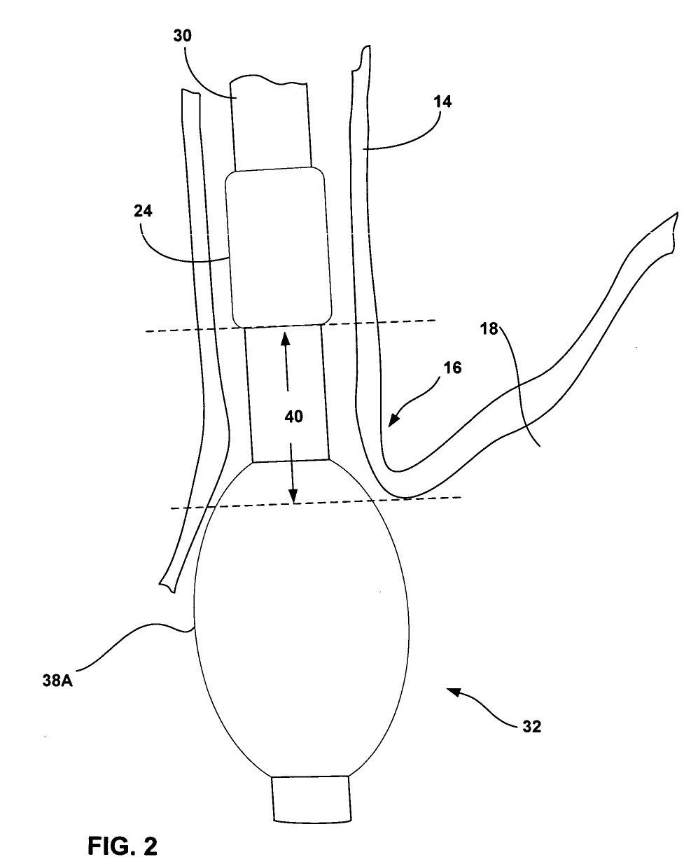

FIG. 2 is a schematic diagram illustrating placement of a distal fixation balloon 38A of endoscopic delivery device 26 (FIG. 1). Endoscopic delivery device 26 is sized for introduction into esophagus 14 via either nasal cavity 34 or oral cavity 36. A medical device, indicated generally by reference numeral 24 in FIG. 2, is carried by flexible probe 30 and positioned at a known, fixed distance 40 from a point at which distal fixation balloon 38A abuts LES 16. Balloon 38A represents one example of a fixation element 38, and is initially in a deflated or non-expanded state for introduction into esophagus 14. Upon deployment of distal end 32 of probe 30 below LES 16, a physician activates a liquid or gas supply to inflate balloon 38A and thereby expand the balloon to a diameter larger than the passage defined by LES 16.

Balloon 38A may be made from a variety of conventional, substantially elastic materials, such as silicone, high density polyurethane, flexible polyvinyl chloride, polyethylene, polyester, or other polymeric materials. An internal lumen within endoscopic delivery device 26 has a proximal port coupled to a fluid source, such as a syringe or fluid pump and reservoir, and a distal end coupled to an interior of balloon 38A. In some embodiments, balloon 38A may have an unexpanded diameter of approximately 5 mm to 25 mm, and an expanded diameter of approximately 30 mm to 60 mm, inclusive of the diameter of flexible probe 30, which may be approximately 10 mm to 40 mm. A length of balloon 38A may be on the order of approximately 1 cm to 5 cm. Balloon 38A, as shown in FIG. 2, may have a tapered profile on proximal and distal ends of the balloon.

The inflated balloon 38A abuts LES 16 and anchors flexible probe 30 against upward movement, such that medical device 24 is placed at a precise position relative to LES 30. In some embodiments, the physician may pull upward on probe 30 to ensure firm engagement of balloon 38A against LES 16. Medical device 24 can then be activated, detached, or otherwise deployed within esophagus 14 to perform an intended diagnostic or therapeutic procedure. As an example, for diagnostic applications such as pH monitoring or manometry, fixed distance 40 may be in the range of approximately 1 cm to 10 cm, and more particularly 3 cm to 7 cm. The pH monitoring capsule in the Medtronic Bravo system, for example, is ordinarily placed at approximately 5 cm from LES 16. A fixed distance 40 of approximately 3 cm to 7 cm, as described herein, should ensure that the pH measurements are taken within a sufficient distance of LES 16 to provide accurate readings. For other applications, including therapeutic applications such as thermal heating or ablation, the fixed distance 40 may be selected according to the requirements of a particular procedure or therapy. For example, thermal heating or ablation may be performed much closer to LES 16 or within LES 16. Following a sufficient course of diagnosis or therapy by medical device 24, or upon detachment or deployment of the medical device, fixation balloon 38A is deflated to permit withdrawal of endoscopic delivery device 26 from esophagus 14.

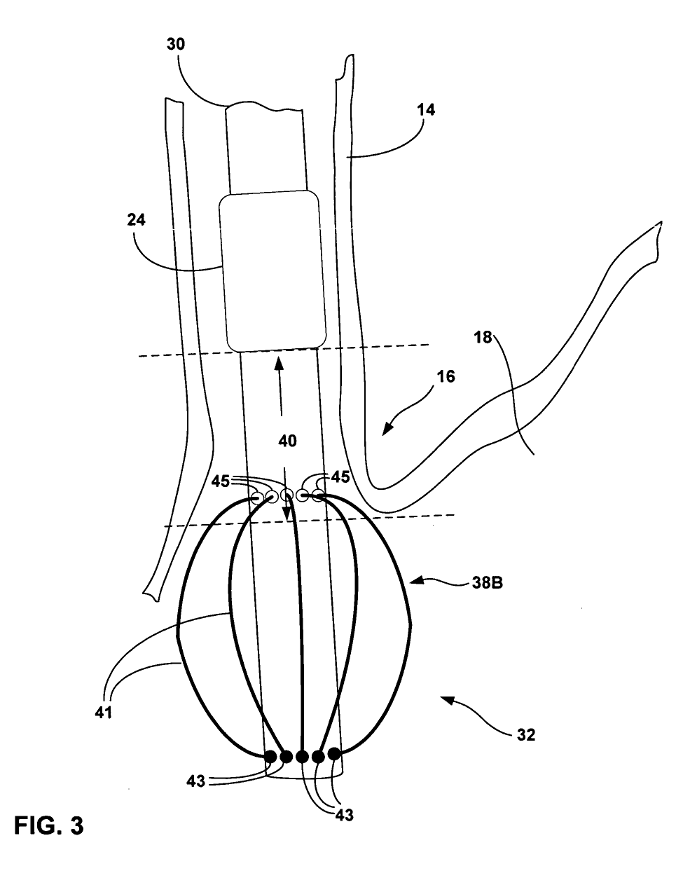

FIG. 3 is a schematic diagram illustrating placement of a distal fixation frame 38B of an endoscopic delivery device 26 for placement of a medical device 24 within esophagus 14. Fixation frame 38B is an example of another type of fixation element 38 suitable of precise, indexed placement of a medical device 24. As an alternative to a balloon 38A (FIG. 2), fixation frame 38B may be constructed in a variety of ways to approximate a basket or other frame-like structure that is expandable to a size larger than a passage defined by LES 16.

In the example of FIG. 3, frame 38B includes a plurality of wire-like members 41 that can be retracted from and withdrawn into endoscopic delivery device 26. Each wire-like member 41 has a distal end fixed to endoscopic delivery device 26 at respective fixation points 43, e.g., by welding, adhesive bonding, crimping or the like. A proximal end of each wire-like member 41 extends into a respective interior lumen 45 defined by endoscopic delivery device 26 such that each wire-like member may extend out of and retract into the interior lumen to cause the fixation frame 38B to expand and retract, respectively. For example, the physician may use a trigger or handle to actuate wire-like members 41, or to actuate coupling rods coupled between the wire-like elements and a proximal end of flexible probe 30. Upon expansion, fixation frame 38B may be sized on the order of fixation balloon 38A of FIG. 2.

As in the example of FIG. 2, medical device 24 is positioned at a known, fixed distance 40 from a point at which fixation frame 38B engages LES 16. Fixation frame 38B, including wire-like members 41, may be constructed from a variety of substantially elastic or in-elastic biocompatible materials, including titanium, stainless steel, shape memory alloys such as nitinol, or the like. However, it is desirable that the material used to form fixation frame 38B should have sufficient structural integrity to resist substantial deformation when the frame abuts LES 16, thereby ensuring that the frame properly anchors endoscopic delivery device 26 against substantial upward movement within esophagus 14.

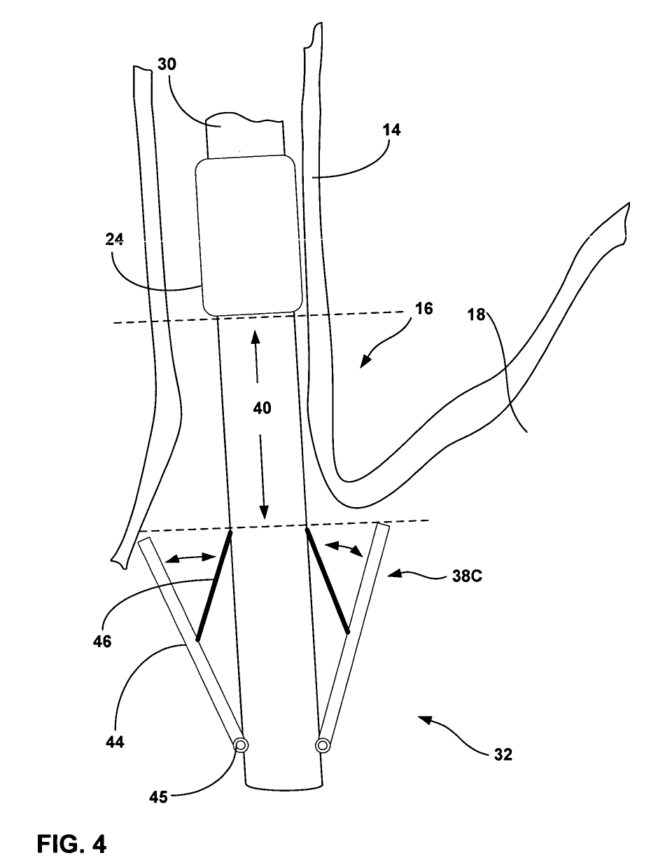

FIG. 4 is a schematic diagram illustrating placement of distal fixation tines 38C of an endoscopic delivery device 26 for placement of a medical device 24 within esophagus 14. Fixation tines 38C, like fixation balloon 38A (FIG. 2) and fixation frame 38B (FIG. 3), serve as a fixation element 38 to anchor endoscopic delivery device 26 relative to LES 16. In this manner, fixation tines 38C permit indexed placement of medical device 24 at a precision position within esophagus 14. As shown in FIG. 4, fixation tines 38C may include one or more individual tines 44 that are expandable outward from flexible probe 30 to a size that is larger than a passage defined by LES 16. Individual tines 44 may be constructed from a variety of materials including titanium, stainless steel, or other biocompatible metals, as well as biocompatible polymeric materials.

Fixation tines 38C include individual tines 44 coupled to respective hinge members 45, and wires 46 to actuate tines 44 inward and outward about the hinge members. Wires 46 may extend along the length of endoscopic delivery device 26 so that a physician may advance and retract the wires to advance and retract tine members 44. When tines 44 are expanded outward, they are able to abut a surface of LES 16 and anchor flexible probe 30 against substantial upward movement relative to the LES. Upon retraction of tines 44 radially inward, endoscopic delivery device 26 can be withdrawn from esophagus 14 by the physician. As discussed above, medical device 24 may remain within esophagus 14 or be withdrawn with flexible probe 30.

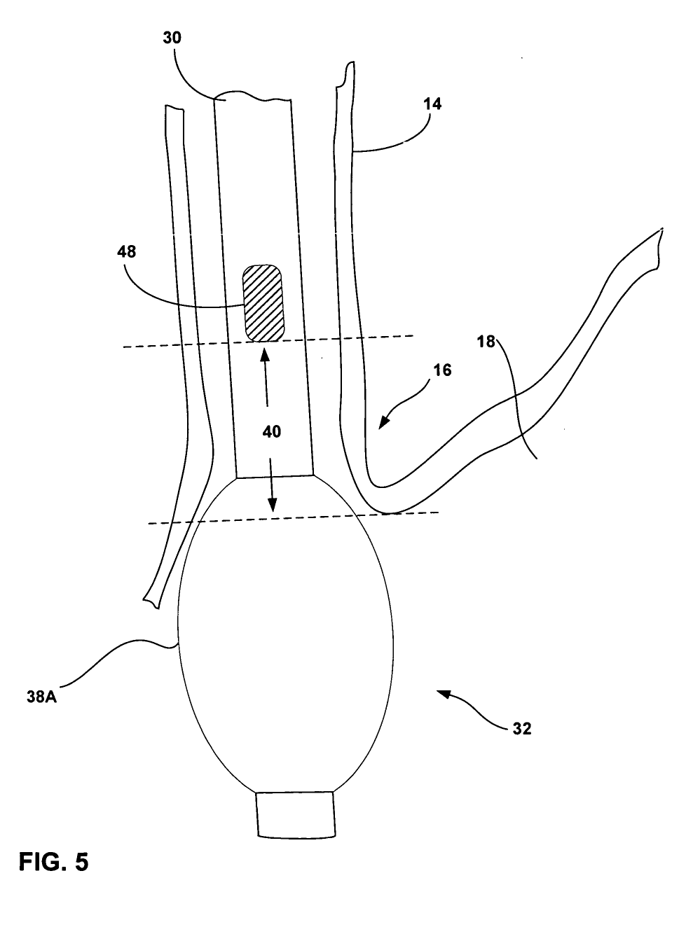

FIG. 5 is a schematic diagram illustrating placement of a distal fixation balloon 38A of an endoscopic delivery device carrying a monitor 48. Hence, in the example of FIG. 5, the medical device takes the form of monitor 48, which may be configured to monitor a variety of physiological parameters within esophagus 14. For example, monitor 48 may be a gastro-esophageal reflux sensor. In particular, monitor 48 may be configured to monitor pH levels proximate LES 16 and thereby support diagnosis of GERD or other gastrointestinal disorders. In other embodiments, monitor 48 may monitor other physiological conditions such as pressure, fluid flow, temperature, or other physiological conditions.

Monitor 48 is carried by flexible probe 30 of endoscopic delivery device 26 for indwelling monitoring applications. In other words, while monitor 48 is activated for use, flexible probe 30 of endoscopic delivery device 26 remains in place within esophagus. Upon completion of a desired course of monitoring, flexible probe 30, as well as monitor 48, are withdrawn from esophagus 14. Upon expansion, balloon 38A serves to anchor flexible probe 30 relative to LES 16, and thereby position monitor 48 at an appropriate location within esophagus 14. Although balloon 38A is shown in FIG. 5 for purposes of illustration, other fixation elements such as those described herein, e.g., in FIGS. 3 and 4, may be used.

In the example of FIG. 5, monitor 48 presents a pH monitor that is mounted within flexible probe 30 and exposed to esophageal contents via a window defined by a wall of the flexible probe. Monitor 48 may be coupled to external processing and storage hardware via one or more cables that extend along the length of flexible probe 30. Alternatively, monitor 48 may be equipped with suitable wireless telemetry circuitry for wireless communication with external hardware. Wireless telemetry may be accomplished by radio frequency communication or proximal inductive interaction of an external controller with monitor 48.

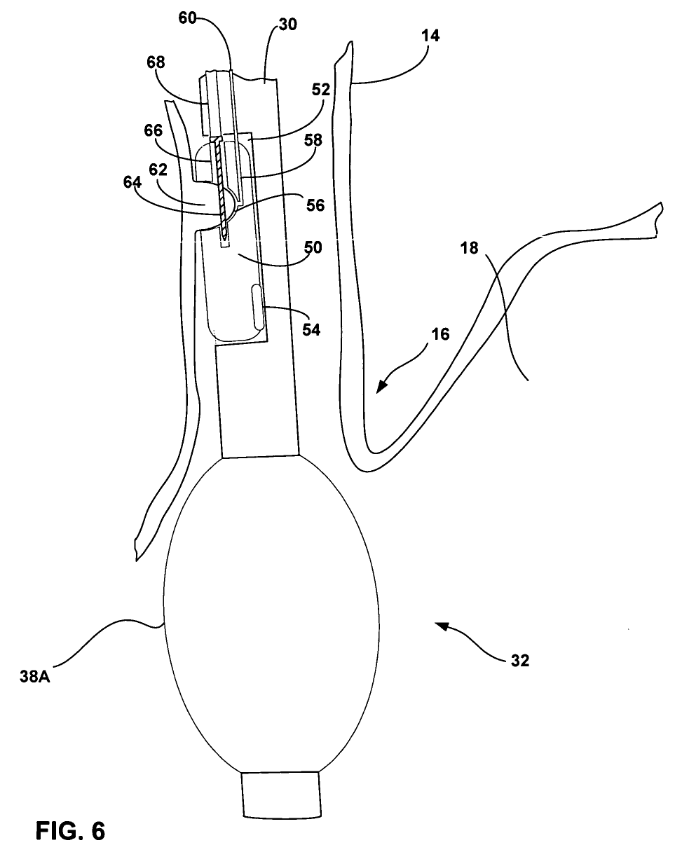

FIG. 6 is a schematic diagram illustrating placement of a distal fixation balloon 38A of an endoscopic delivery device 26 carrying a detachable monitor 50. In the example of FIG. 6, endoscopic delivery device 26 and distal fixation balloon 38 are used for precise placement of a monitor 50 that is detached from flexible probe 30 and then attached to a mucosal lining of esophagus 14 above LES 16. Detachable monitor 50 may have a capsule-like device housing. In particular, detachable monitor 50 may conform substantially to detachable monitors described in commonly assigned U.S. Pat. Nos. 6,285,897 and 6,698,056 to Kilcoyne et al. provide examples of fixation mechanisms for attaching monitoring devices to the lining of esophagus 14. The contents of the Kilcoyne et al. patents are incorporated herein by reference in their entireties.

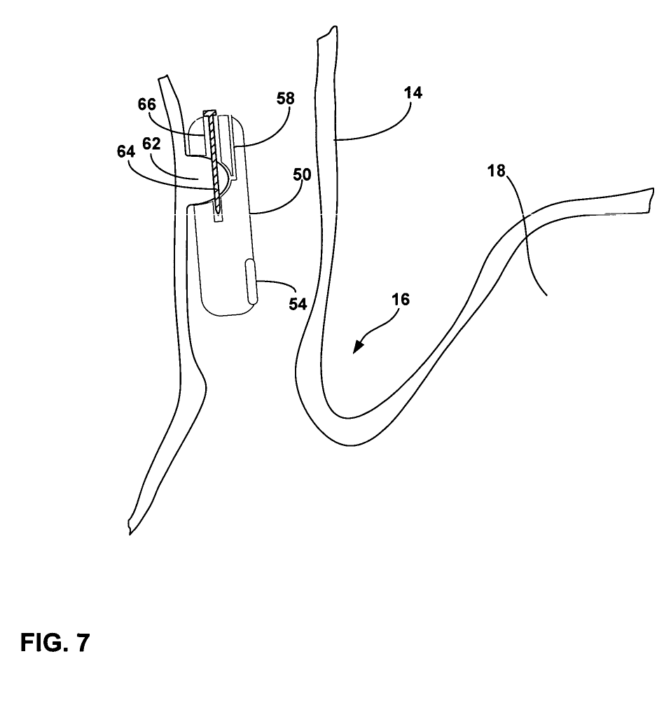

As shown in FIG. 6, flexible probe 30 defines a monitor placement bay 52 to hold monitor 50 for deployment to a desired position. Monitor placement bay 52 serves as a deployment point, formed within flexible probe 30, for deployment of monitor 50 at a fixed distance from distal fixation balloon 38A. A sensor 54 is carried by monitor 50 at a position exposed to esophageal contents. Monitor 50 defines a vacuum cavity 56 coupled to a vacuum line 58. A vacuum line 60 in flexible probe 30 applies vacuum pressure to vacuum cavity 56 via vacuum line 58 to draw a portion 62 of esophageal tissue into the vacuum cavity. Then, a pin 64 is driven into portion 62 of esophageal tissue, e.g., by advancing a control rod 68 within flexible probe 30. Pin 64 penetrates the esophageal tissue, and thereby attaches monitor 50 to esophagus 14 for prolonged monitoring of conditions such as pH levels. FIG. 7 is a schematic diagram illustrating the monitor 50 of FIG. 6 upon detachment from endoscopic delivery device 26.

In some embodiments, the capsule-like device housing of monitor 50 may have a maximum length of less than approximately 10 mm and a maximum width of less than approximately 5 mm. The capsule-like device housing may be substantially cylindrical, with a length greater than its diameter and flat or rounded ends, although the invention is not limited to any particular shape. For a cylindrical device housing, monitor 50 may have a maximum height of less than approximately 10 mm and a maximum diameter of less than approximately 5 mm. The housing for monitor 50 may be formed from a variety of biocompatible materials such as stainless steel or titanium.

As described in the Kilcoyne patents, the capsule-like device housing of monitor 50 further includes a power source, a monitor, signal processing electronics, and a fixation mechanism, e.g., pin 64, to attach the monitor to the mucosal lining of esophagus 14. The fixation mechanism may perforate the mucosa and lodge in the muscularis external of the gastrointestinal tract wall when introduced against the mucosa, or grip a fold of the mucosa. The fixation mechanism may take a variety of alternative forms, and may include a variety of features such as one or more shafts, hooks, barbs, screws, sutures, clips, pincers, staples, tacks, or other fasteners.

In some embodiments, the fixation mechanism can at least partially penetrate the mucosal lining of the gastrointestinal tract. In other embodiments, the fixation mechanism pinches or otherwise holds a fold of mucosal lining tissue. In either case, the fixation mechanism securely attaches monitor 50 to the target location, subject to detachment when mucosal tissue sloughs away to release the monitor. In some embodiments, the fixation mechanism may be made from a degradable material that degrades or absorbs over time at the attachment site to release monitor 50 from tissue at the target location. In either case, upon detachment, monitor 50 passes through the gastrointestinal tract of patient 12. The Kilcoyne et al. patents provide examples of fixation mechanisms for attaching monitoring devices to the lining of the esophagus, including suitable degradable materials.

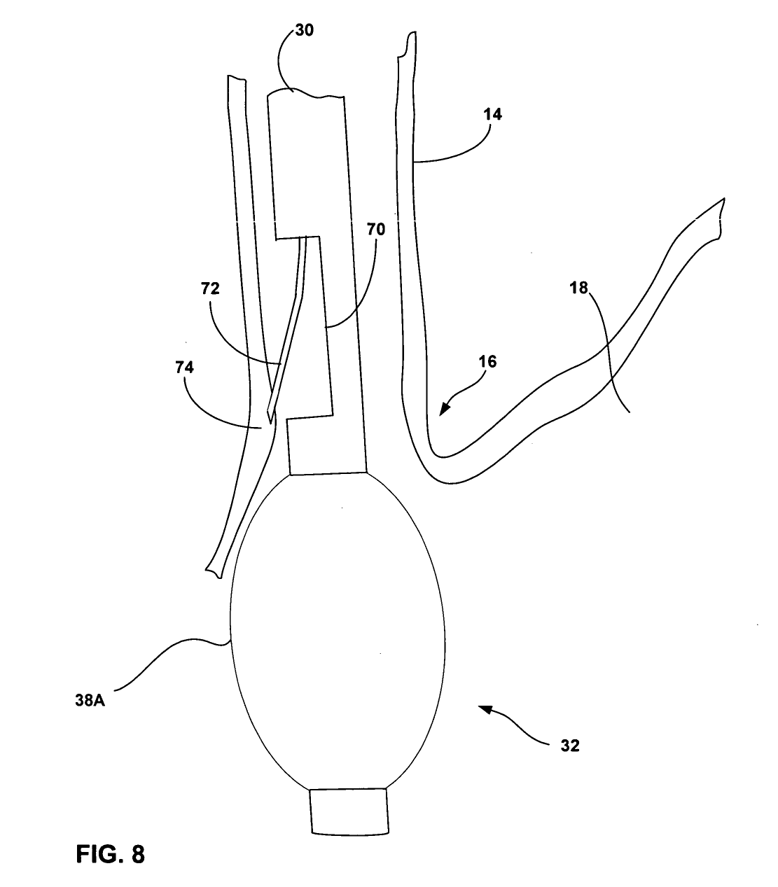

FIG. 8 is a schematic diagram illustrating placement of a distal fixation balloon 38A of an endoscopic delivery device 26 carrying components for implantation of bulking agents in the esophageal wall. Commonly assigned U.S. Pat. No. 6,401,718 to Johnson et al. describes methods and devices for delivery and placement of bulking agents within the esophageal wall adjacent LES 16 to treat GERD. Endoscopic delivery device 26 may incorporate components similar to those described in the Johnson et al. patent, which is incorporated herein by reference in its entirety. For example, flexible probe 30 may define an opening 70, which serves as a deployment point for deployment of one or more medical devices in the form of endoscopic instruments, such as needle 72, to prepare a portion 74 of the esophageal wall for placement of a bulking agent.

Upon placement of expandable fixation balloon 38A, or other alternative fixation elements, flexible probe 30 is positioned precisely relative to LES 16. As described in the Johnson et al. patent, a needle 72 may be used to define a pocket for placement of the bulking agent. For example, needle 72 may deliver saline or another injectable substance into portion 74 of the esophageal wall to provide an enlarged receiving pocket for the bulking agent. A physician endoscopically deploys needle 72 from opening 70 of flexible probe 30.

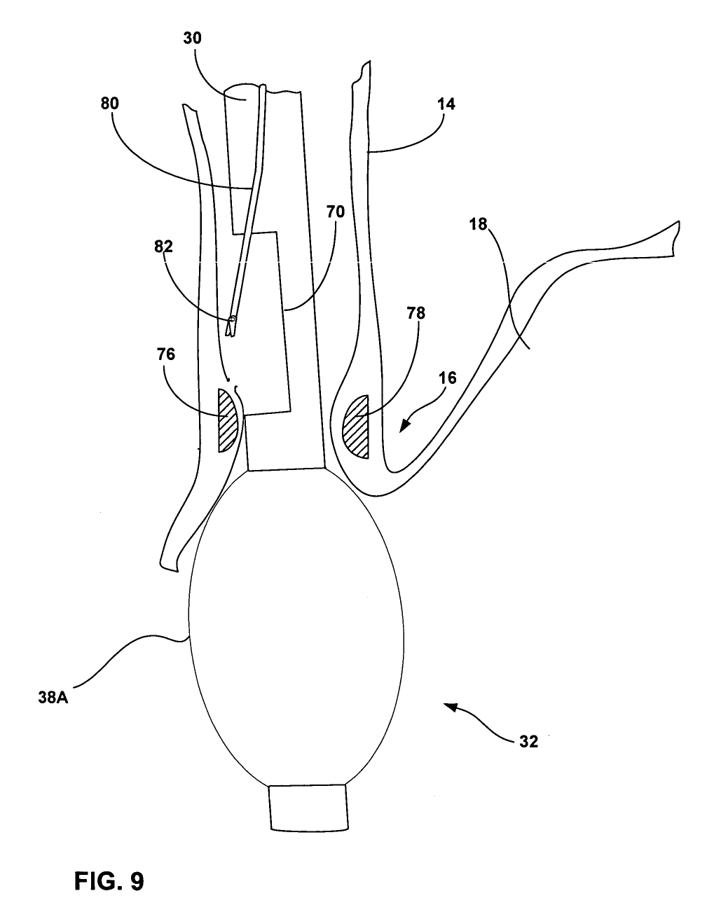

FIG. 9 is a schematic diagram of the endoscopic delivery device 26 of FIG. 8 following implantation of bulking agents in the esophageal wall. As shown in FIG. 9, upon withdrawal of needle 72, an endoscopic instrument 80 with a gripping member 82, e.g., pincers or jaws, is used to deliver and place endoscopic bulking agents 76, 78 within respective pockets in the wall of esophagus 14. The bulking agents 76, 78 may include dehydrated hydrogel materials that tend to take on water and swell in size upon implantation, or other materials as described in the Johnson et al. patent. In each case, bulking agent 76, 78 may serve as part of a GERD therapy by enhancing closing pressure of LES 16 to prevent or reduce reflux.

As illustrated in FIGS. 8 and 9, a distal fixation element such as expandable fixation balloon 38A serves to anchor flexible probe 30 and thereby permit precise placement of bulking agents 76, 78. In particular, the known, fixed distance between the point of contact of balloon 38A and LES 16 and the position of opening 70 facilitates quick and accurate positioning. The positioning step may be aided by conventional endoscopic viewing or external imaging, but is significantly simplified by the use of an expandable fixation element for indexed positioning.

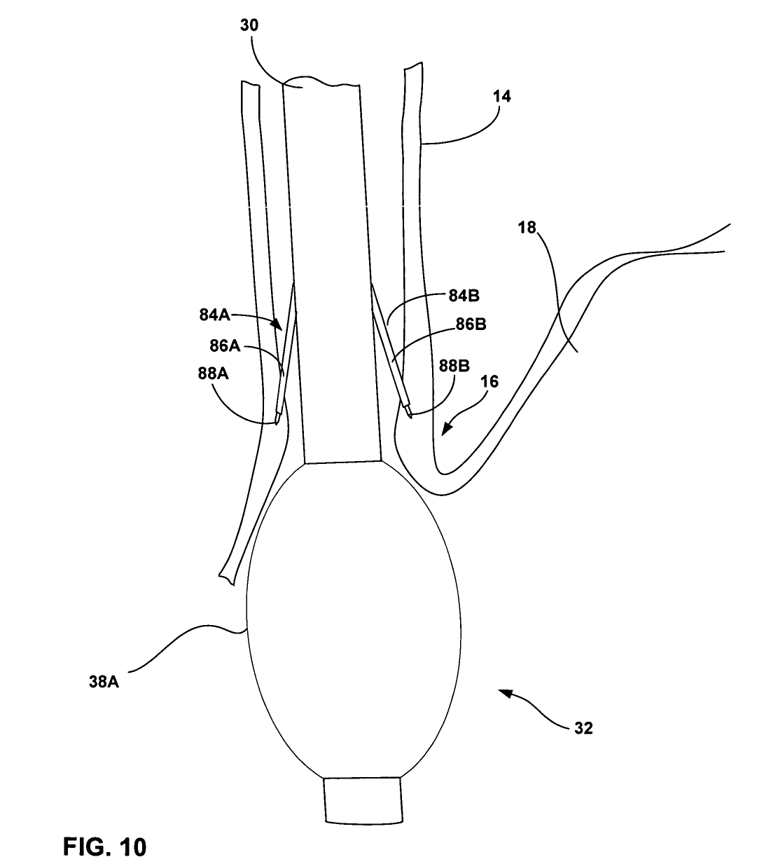

FIG. 10 is a schematic diagram illustrating placement of a distal fixation balloon 38A of an endoscopic delivery device 26 carrying components for heat treatment of esophageal tissue. As shown in FIG. 10, flexible probe 30 of endoscopic delivery device 26 carries one or more radio frequency (RF) needle assemblies 84A, 84B (collectively 84). In the example of FIG. 10, needle assemblies 84 are carried at a deployment point at a fixed distance from a fixation balloon 38A. Each needle assembly 84 may include a respective insulative sleeve 86A, 86B and a respective conductive needle 88A, 88B. Conductive needles 88 are coupled to electrical conductors that extend along the length of flexible probe 30. Upon expansion of balloon 38A to anchor flexible probe 30, a physician advances the electrical conductors to extend needle assemblies 84 outward from the flexible probe for penetration of esophageal tissue in the vicinity of LES 16.

Needle assemblies 84A, 84B are retractable into flexible probe 30 for deployment of endoscopic delivery device 26 within esophagus 14, and extendable for contact with esophageal tissue. Needle assemblies 84A, 84B may contact or penetrate tissue adjacent to or within LES 16. In either case, conductive needles 88A, 88B are capable of transmitting RF energy into tissue lining esophagus 14. The physician activates an RF current source to drive a selected amount of RF energy for a selected duration into the tissue via needles 88. A reference electrode may be attached to the body of patient 12 to complete the electrical circuit with needles 88.

The RF energy generated by needles 88 serves to shrink collagen within the tissue to reduce the size of LES 16. Following delivery of a sufficient amount of RF energy, the physician may retract needle assemblies 84A, 84B into flexible probe 30 for withdrawal of endoscopic delivery device 26 from the patient. Alternatively, in some embodiments, the physician may retract needle assembles 84A, 84B, rotate flexible probe 30 within esophagus 14, and then advance needle assemblies 84A, 84B to access other portions of the tissue adjacent LES 16. In some embodiments, flexible probe 30 may incorporate one, two, three or more needle assemblies to simultaneously access multiple tissue sites adjacent to or within LES 16. Also, in other embodiments, needle assemblies 84A, 84B may be positioned to access other locations within esophagus 14, further away from LES 16.

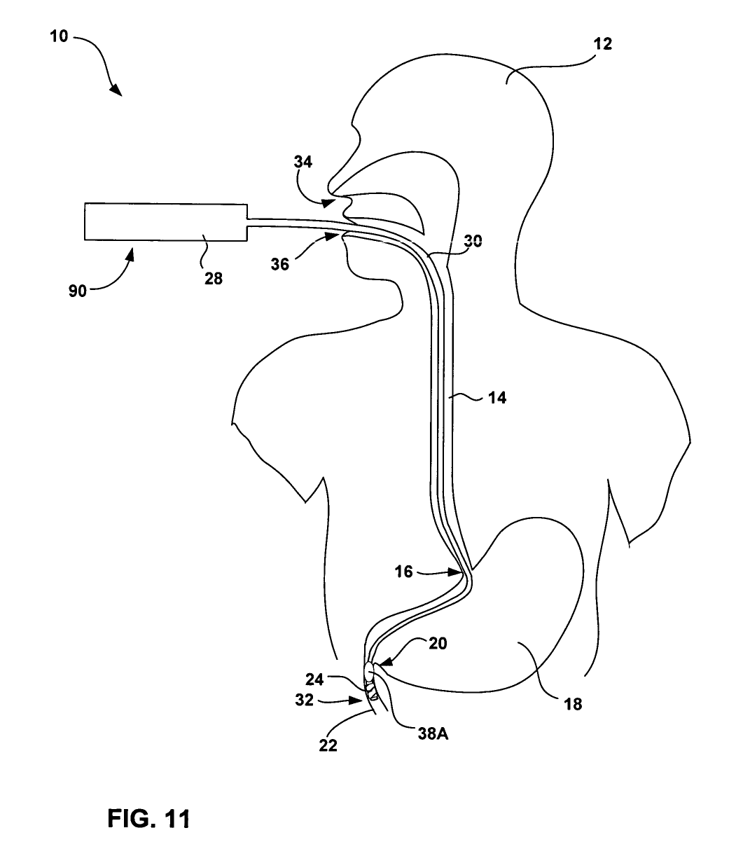

FIG. 11 is a schematic diagram illustrating an esophageal delivery system 90 for deployment of a medical device 24 in the small intestine 22 proximate the pyloric sphincter 20, shown in conjunction with a patient 12. Esophageal delivery system 90 conforms substantially to system 10 of FIG. 1. Accordingly, like reference numerals are used to refer to like components within systems 90 and 10. For example, system 90 includes handle 28, flexible probe 30, and fixation balloon 38A. Flexible probe 30 extends into esophagus 14 via nasal passage 34 or oral passage 36. However, esophageal delivery system 90 is further designed to extend through stomach 18 and pyloric sphincter 20 such that a distal end 32 of flexible probe 30 enters small intestine 22.

As shown in FIG. 11, distal end 32 of flexible probe 30 includes a fixation element to anchor the distal end relative to pyloric sphincter 20. In the example of FIG. 11, the fixation element is fixation balloon 38A. In other embodiments, however, the fixation element may take the form of an expandable frame, expandable tines or the like, as described herein. In each case, the fixation element is sized larger than a passage defined by pyloric sphincter 20. Upon entry of distal end 32 of flexible probe 30 into stomach 18, a physician expands fixation balloon 38A.

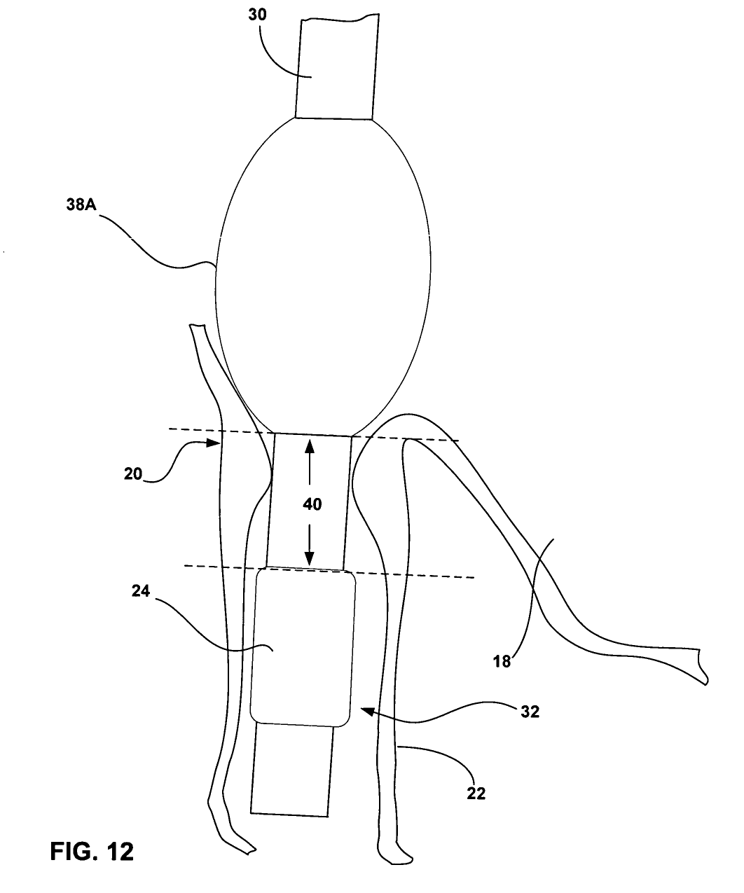

Fixation balloon 38A is expanded following passage of distal end 32 into pyloric sphincter 20, but prior to passage of the fixation element through the pyloric sphincter. In this manner, the fixation element abuts pyloric sphincter to resist substantial downward movement of flexible probe 30 through pyloric sphincter 20. FIG. 12 is a schematic diagram further illustrating placement of distal fixation balloon 38A of endoscopic delivery device 90 proximate pyloric sphincter 20. Fixation balloon 38A anchors flexible probe 30 against substantial downward movement and thereby provides a reference position that is indexed to the position of pyloric sphincter 20.

A medical device 24 is positioned at deployment point situated at a known, fixed distance 40 from the point at which fixation balloon 38A abuts pyloric sphincter 20, as in other embodiments. However, medical device 24 is positioned on a side of fixation balloon 38A adjacent the distal tip of flexible probe 30. As a result, medical device 24 is positioned within small intestine 22 on a side of pyloric sphincter 20 opposite fixation balloon 38A. Moreover, medical device 24 is placed at a precise position that is indexed to the position of pyloric sphincter 20, as a result of the fixed distance 40 between fixation balloon 38A and the medical device. As an example, fixed distance 40 in FIG. 12 may be on the order of approximately 2 cm to 15 cm, depending on the desired application.

Although medical device 24 is indicated generally in FIGS. 11 and 12, esophageal delivery system 90 may be equipped with any of a variety of diagnostic or therapeutic medical devices suitable for use within the small intestine in the region proximate the pyloric sphincter. As examples, the medical device may comprise a pH monitor, flow, pressure, or temperature sensors, components for placement of a bulking device, a stimulator, a drug delivery device, a thermal element to treat tissue in the vicinity of the pyloric sphincter, or other types of devices.

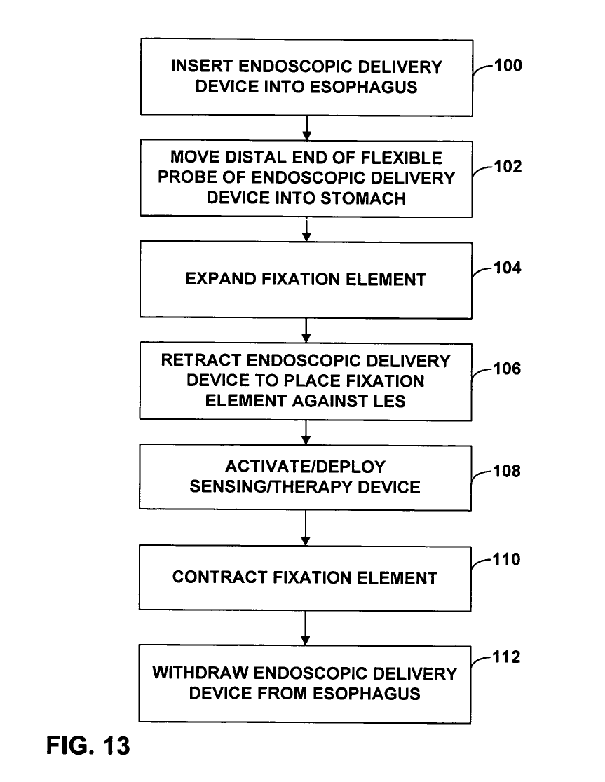

FIG. 13 is a flow diagram illustrating a method for placement of a medical device within the esophagus, as described herein. As shown in FIG. 13, the method involves inserting an endoscopic delivery device into the esophagus (100), and moving the distal end of a flexible probe forming part of the endoscopic delivery device into the stomach (102). Upon expansion of a fixation element on the stomach side of the LES (104), the flexible probe is retracted to place the fixation element against the LES (106) and thereby anchor the probe against substantial upward movement. At this point, a medical device carried by the flexible probe is placed at a known, indexed position relative to the LES.

The known position corresponds to a desired therapy or diagnostic location within the esophagus. Accordingly, a physician activates or deploys a therapeutic or diagnostic medical device (108). Upon completion of a desired course of therapy or diagnosis, or following deployment of a medical device, the fixation element is contracted (110), and the endoscopic delivery device is withdrawn from the esophagus (112). In some embodiments, the medical device is withdrawn with the endoscopic delivery device. In other embodiments, the medical device remains within the esophagus.

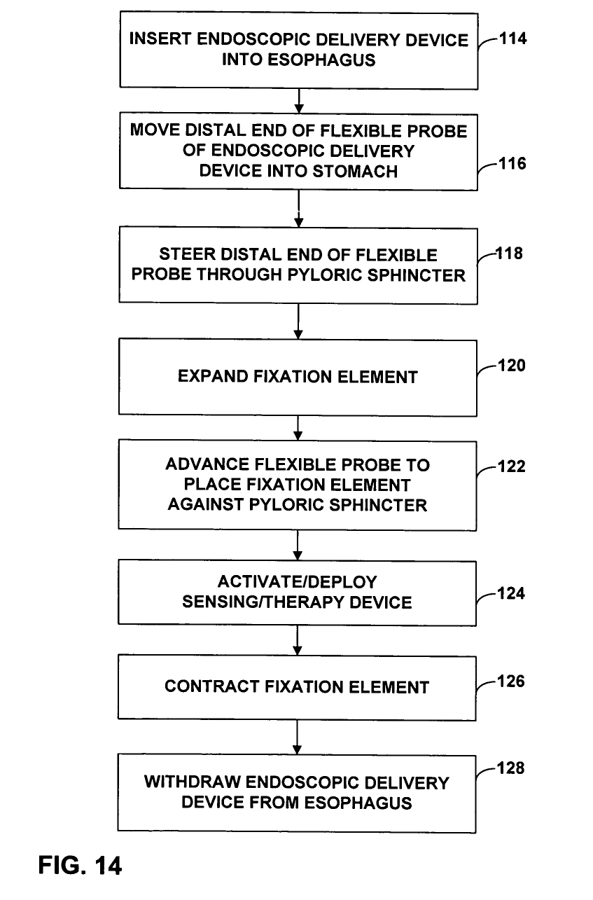

FIG. 14 is a flow diagram illustrating a method for placement of a medical device within the small intestine adjacent the pyloric sphincter. The method depicted in FIG. 14 substantially conforms to the method of FIG. 13, but involves placement of a medical device within the small intestine rather than the esophagus. As shown in FIG. 14, the method may involve inserting an endoscopic delivery device into the esophagus (114), moving a distal end of a flexible probe associated with the endoscopic delivery device into the stomach (116), and steering a distal end of the flexible probe through the pyloric sphincter (118). A physician may steer the flexible probe using conventional steering components such as embedded wires, shape memory elements, or the like. Upon expansion of a fixation element carried by the flexible probe (120), the probe is advanced to place the fixation element against the pyloric sphincter and thereby anchor the probe against substantial downward movement (122).

At this point, a medical device at the distal end of the probe is within the small intestine at a known, fixed distance from the point at which the fixation element contacts the pyloric sphincter. A physician activates or deploys the diagnostic or therapy device (124). Upon completion of a desired course of diagnosis or therapy, or deployment of the medical device, the fixation element is contracted (126), and the endoscopic delivery device is withdrawn from the small intestine, stomach and esophagus (128).

The preceding specific embodiments are illustrative of the practice of the invention. It is to be understood, therefore, that other expedients known to those skilled in the art or disclosed herein may be employed without departing from the invention or the scope of the claims. For example, the invention need not be limited to deployment of a medical device at a particular location within the esophagus or small intestine. In various embodiments, a medical device may be located anywhere within the esophagus or small intestine, and take advantage of indexed positioning with the use of a fixation element as described herein.

The invention also is not limited to monitoring devices, bulking devices, electrical stimulators, drug delivery devices, or thermal elements, but also may encompass medical devices configured to deliver different types of therapies or to serve different diagnostic purposes. In addition, the invention is not limited to application for monitoring or therapy applications associated with any particular disorder, condition or affliction.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. An esophageal delivery system comprising:
an elongated delivery device sized for introduction into an esophagus of a patient;
a fixation element, disposed adjacent a distal end of the elongated delivery device, to engage a first selected portion of the esophagus and thereby fix the elongated delivery device against substantial upward movement within the esophagus; and
a medical device for placement within the esophagus of the patient, wherein the medical device is carried by the elongated delivery device at a fixed position relative to the fixation element such that the medical device is positioned adjacent a second selected portion of the esophagus.

2. The system of claim 1, wherein the fixation element comprises an expandable balloon that, upon expansion, is sized larger than a passage defined by a lower esophageal sphincter of the patient.

3. The system of claim 1, wherein the fixation element comprises an expandable frame that, upon expansion, is sized larger than a passage defined by a lower esophageal sphincter of the patient.

4. The system of claim 1, wherein the fixation element comprises one or more expandable tines that, upon expansion, are oriented to engage tissue in the vicinity of the lower esophageal sphincter.

5. The system of claim 1, wherein the medical device is disposed at a side of the fixation element opposite a distal tip of the elongated delivery device.

6. The system of claim 1, wherein the medical device comprises a gastro-esophageal reflux sensor.

7. The system of claim 1, wherein the gastro-esophageal reflux sensor comprises a pH sensor.

8. The system of claim 1, wherein the medical device comprises a device for introducing a bulking agent into a wall of the esophagus.

9. The system of claim 8, wherein the medical device comprises one or more endoscopic members to prepare the wall of the esophagus for introduction of the bulking agent and introduce the bulking agent.

10. The system of claim 8, wherein the bulking agent comprises an expandable hydrogel implant.

11. The system of claim 1, wherein the medical device comprises a heating element to heat tissue adjacent a lower esophageal sphincter of the patient.

12. The system of claim 11, wherein the heating element comprises an electrically conductive probe to deliver radio frequency energy to the tissue.

13. The system of claim 1, wherein the medical device comprises an electrical stimulator or a drug delivery device.

14. A gastrointestinal delivery system comprising: an elongated delivery device sized for introduction into a small intestine of a patient via an esophagus of the patient;
a fixation element, disposed adjacent a distal end of the elongated delivery device, to engage tissue adjacent a pyloric sphincter of the patient, and thereby fix the elongated delivery device against substantial downward movement within the small intestine; and
a medical device for placement within the small intestine of the patient, wherein the medical device is carried by the elongated delivery device at a fixed position relative to the fixation element such that the medical device is positioned adjacent a selected portion of the small intestine below the pyloric sphincter.

15. The system of claim 14, wherein the fixation element comprises an expandable balloon that, upon expansion, is sized larger than a passage defined by the pyloric sphincter of the patient.

16. The system of claim 14, wherein the fixation element comprises an expandable frame that, upon expansion, is sized larger than a passage defined by the pyloric sphincter of the patient.

17. The system of claim 14, wherein the fixation element comprises one or more expandable tines that, upon expansion, are oriented to engage tissue in the vicinity of the pyloric sphincter.

18. The system of claim 14, wherein the medical device is disposed at a side of the fixation element adjacent a distal tip of the elongated delivery device.

19. The system of claim 14, wherein the medical device comprises a diagnostic sensor.

20. The system of claim 14, wherein the medical device comprises a device for introducing a bulking agent into a wall of the small intestine.

21. The system of claim 14, wherein the medical device comprises a heating element to heat tissue adjacent a pyloric sphincter of the patient.

22. An esophageal delivery system comprising:
a medical device;
means for introducing the medical device into an esophagus of a patient; and
means for engaging a first selected portion of the esophagus to fix the introducing means against substantial upward movement within the esophagus, wherein the medical device is carried by the introducing means at a fixed position relative to the engaging means such that the medical device is positioned adjacent a second selected portion of the esophagus.

23. The system of claim 22, wherein the engaging means comprises an expandable balloon that, upon expansion, is sized larger than a passage defined by a lower esophageal sphincter of the patient.

24. The system of claim 22, wherein the engaging means comprises an expandable frame that, upon expansion, is sized larger than a passage defined by a lower esophageal sphincter of the patient.

25. The system of claim 22, wherein the engaging means comprises one or more expandable tines that, upon expansion, are oriented to engage tissue in the vicinity of the lower esophageal sphincter.

26. The system of claim 22, wherein the medical device comprises means for sensing gastro-esophageal reflux.

27. The system of claim 22, wherein the medical device comprises means for introducing a bulking agent into a wall of the esophagus.

28. The system of claim 22, wherein the medical device comprises means for heating tissue adjacent a lower esophageal sphincter of the patient.

29. The system of claim 22, wherein the medical device comprises one of a means for electrically stimulating tissue within the esophagus and a means for delivering a drug.

30. A gastro-intestinal delivery system comprising:
a medical device;
means for introducing the medical device into a small intestine of a patient via an esophagus of the patient; and
means for engaging tissue adjacent a pyloric sphincter of the patient to fix the introducing means against substantial downward movement within the small intestine, wherein the medical device is carried by the introducing means at a fixed position relative to the engaging means such that the medical device is positioned adjacent a selected portion of the small intestine below the pyloric sphincter.

31. The system of claim 30, wherein the engaging means comprises an expandable balloon that, upon expansion, is sized larger than a passage defined by the pyloric sphincter of the patient.

32. The system of claim 30, wherein the engaging means comprises an expandable frame that, upon expansion, is sized larger than a passage defined by the pyloric sphincter of the patient.

33. The system of claim 30, wherein the engaging means comprises one or more expandable tines that, upon expansion, are oriented to engage tissue in the vicinity of the pyloric sphincter.

34. The system of claim 30, wherein the medical device comprises a diagnostic sensor.

35. The system of claim 30, wherein the medical device comprises a device for introducing a bulking agent into a wall of the small intestine.

36. The system of claim 30, wherein the medical device comprises a heating element to heat tissue adjacent a pyloric sphincter of the patient.

37. A method for esophageal delivery of a medical device, the method comprising:
introducing an elongated delivery device into an esophagus of a patient;
engaging a first selected portion of the esophagus with a fixation element carried by the elongated delivery device to thereby fix the elongated delivery device against substantial upward movement within the esophagus; and
placing a medical device within the esophagus of the patient, wherein the medical device is carried by the elongated delivery device at a fixed position relative to the fixation element such that the medical device is positioned adjacent a second selected portion of the esophagus.

38. The method of claim 37, further comprising extending the fixation element to a position below the lower esophageal sphincter, and pulling the elongated delivery device upward to engage the fixation element with the lower esophageal sphincter.

39. The method of claim 38, wherein the fixation element comprises an expandable balloon that, upon expansion, is sized larger than a passage defined by a lower esophageal sphincter of the patient, the method further comprising:
extending the fixation element to a position below the lower esophageal sphincter;
expanding the balloon; and
pulling the elongated delivery device upward to engage the expanded balloon with the lower esophageal sphincter

40. The method of claim 38, wherein the fixation element comprises an expandable frame that, upon expansion, is sized larger than a passage defined by a lower esophageal sphincter of the patient, the method further comprising:
extending the fixation element to a position below the lower esophageal sphincter;
expanding the frame; and
pulling the elongated delivery device upward to engage the expanded frame with the lower esophageal sphincter.

41. The method of claim 38, wherein the fixation element comprises one or more expandable tines that, upon expansion, are oriented to engage tissue in the vicinity of the lower esophageal sphincter, the method further comprising:
extending the fixation element to a position below the lower esophageal sphincter;
expanding the tines; and
pulling the elongated delivery device upward to engage the expanded tines with the lower esophageal sphincter.

42. The method of claim 37, wherein the medical device is disposed at a side of the fixation element opposite a distal tip of the elongated delivery device.

43. The method of claim 37, wherein the medical device comprises an gastro-esophageal reflux sensor.

44. The method of claim 37, wherein the medical device comprises a device for introducing a bulking agent into a wall of the esophagus.

45. The method of claim 44, wherein the bulking agent comprises an expandable hydrogel implant.

46. The method of claim 37, wherein the medical device comprises a heating element to heat tissue adjacent a lower esophageal sphincter of the patient.

47. The method of claim 37, wherein the medical device comprises one of an electrical stimulator and a drug delivery device.

48. A method for gastro-intestinal delivery of a medical device, the method comprising:
introducing an elongated delivery device sized into a small intestine of a patient via an esophagus and a stomach of the patient;
engaging tissue adjacent a pyloric sphincter of the patient with a fixation element to fix the elongated delivery device against substantial downward movement within the small intestine; and
placing a medical device within the small intestine of the patient, wherein the medical device is carried by the elongated delivery device at a fixed position relative to the fixation element such that the medical device is positioned adjacent a selected portion of the small intestine below the pyloric sphincter.

49. The method of claim 48, further comprising extending the fixation element to a position above the pyloric sphincter, and pushing the elongated delivery device downward o engage the fixation element with the pyloric sphincter.

50. The method of claim 49, wherein the fixation element comprises an expandable balloon that, upon expansion, is sized larger than a passage defined by a pyloric sphincter of the patient, the method further comprising:
extending the fixation element to a position above the pyloric sphincter;
expanding the balloon; and
pushing the elongated delivery device downward to engage the expanded balloon with the pyloric sphincter

51. The method of claim 49, wherein the fixation element comprises an expandable frame that, upon expansion, is sized larger than a passage defined by the pyloric sphincter of the patient, the method further comprising:
extending the fixation element to a position above the pyloric sphincter;
expanding the frame; and
pushing the elongated delivery device downward to engage the expanded frame with the pyloric sphincter.

52. The method of claim 49, wherein the fixation element comprises one or more expandable tines that, upon expansion, are oriented to engage tissue in the vicinity of the pyloric sphincter, the method further comprising:
extending the fixation element to a position above the pyloric sphincter;
expanding the tines; and
pushing the elongated delivery device downward to engage the expanded tines with the pyloric sphincter.

53. The method of claim 48, wherein the medical device is disposed at a side of the fixation element adjacent a distal tip of the elongated delivery device.

54. The method of claim 48, wherein the medical device comprises an gastro-esophageal reflux sensor.

55. The method of claim 48, wherein the medical device comprises a device for introducing a bulking agent into a wall of the esophagus.

56. The method of claim 48, wherein the medical device comprises a heating element to heat tissue adjacent a lower esophageal sphincter of the patient.

57. The method of claim 48, wherein the medical device comprises one of an electrical stimulator and a drug delivery device.

58. An esophageal delivery system comprising: an elongated delivery device sized for introduction into an esophagus of a patient;
a fixation element, disposed adjacent a distal end of the elongated delivery device, to engage a first selected portion of the esophagus and thereby fix the elongated delivery device against substantial upward movement within the esophagus; and
a deployment point formed in the elongated delivery device to deploy a medical device within the esophagus of the patient, wherein the deployment point is formed in the elongated delivery device at a fixed position relative to the fixation element such that the medical device is deployable to a second selected portion of the esophagus.

59. The system of claim 59, wherein the fixation element comprises an expandable balloon, an expandable frame, or one or more expandable tines that, upon expansion, is oriented to engage tissue in the vicinity of the lower esophageal sphincter.

60. A gastrointestinal delivery system comprising:
an elongated delivery device sized for introduction into a small intestine of a patient via an esophagus of the patient;
a fixation element, disposed adjacent a distal end of the elongated delivery device, to engage tissue adjacent a pyloric sphincter of the patient, and thereby fix the elongated delivery device against substantial downward movement within the small intestine; and
a deployment point formed within the elongated delivery device to deploy a medical device within the small intestine of the patient, wherein the deployment point is formed in the elongated delivery device at a fixed position relative to the fixation element such that the medical device is deployable to a selected portion of the small intestine below the pyloric sphincter.

61. The system of claim 60, wherein the fixation element comprises an expandable balloon that, upon expansion, is sized larger than a passage defined by the pyloric sphincter of the patient.
